(19) United States
(12) Patent Application Publication
(10) Pub. No.: US 2023/0321089 A1
(43) Pub. Date: Oct. 12, 2023

(54) COMBINATION OF CABOTEGRAVIR AND LEVONORGESTREL

(71) Applicant: ViiV Healthcare Company, Wilmington, DE (US)

(72) Inventors: Nima AKHAVEIN, Collegeville, PA (US); Simone ALIDORI, Collegeville, PA (US); Emile Johann VELTHUISEN, Durham, NC (US)

(21) Appl. No.: 18/042,485

(22) PCT Filed: Aug. 30, 2021
(86) PCT No.: PCT/US2021/048127

§ 371 Date: Feb. 22, 2023

Related U.S. Application Data
(60) Provisional application No. 63/073,140, filed on Sep. 1, 2020.

(51) Int. Cl. A61K 31/4985; A61K 31/567; A61K 47/10; A61K 47/26

(52) U.S. Cl. CPC A61K 31/4985; A61K 31/567; A61K 47/10; A61K 47/26

(57) ABSTRACT

The present invention relates to a combination formulation of antiretroviral pharmaceutical compositions and contraceptive agents for the treatment or prevention of human immunodeficiency virus (HIV) and the prevention of pregnancy, the composition comprising an effective amount of Cabotegravir and an effective amount of a contraceptive agent. The present invention also provides a method of preventing pregnancy and treating or preventing HIV in a human by administering a therapeutically effective amount of Cabotegravir and an effective amount of a contraceptive agent to the human.

### FIELD OF THE INVENTION

The present invention relates to a long-acting formulation of a novel combination comprising the integrase strand transfer inhibitor, Cabotegravir or a pharmaceutically acceptable salt or solvate thereof, with a contraceptive agent, pharmaceutical compositions comprising the same and methods of using such combinations and compositions for the dual purpose of preventing pregnancy and preventing or treating HIV infection whilst also lessening the risk of sexually transmitting HIV infection.

### BACKGROUND TO THE INVENTION

Over the past decades, advances in highly active antiretroviral therapies (ARTs) have improved treatment efficacy for patients with human immunodeficiency virus (HIV), improving patient survival and quality of life. However, proper adherence to treatment regimens remains a challenge where poor compliance can result in treatment failure and the emergence of drug-resistant mutations. To help aid adherence, longer acting treatments are under investigation. Both oral and long-acting injectable ART may provide patients with a convenient and discreet approach to manage HIV infection.

Cabotegravir (GSK1265744) is an integrase strand transfer inhibitor (INSTI) that exhibits subnanomolar potency and antiviral activity against a broad range of HIV-1 strains. Oral administration of Cabotegravir has exhibited acceptable safety and tolerability profiles, a long half-life, and few drug-drug interactions. In the phase IIb LATTE trial (ClinicalTrials.gov identifier, NCT01641809), a two-drug regimen of once-daily oral formulations of Cabotegravir and rilpivirine demonstrated durable viral suppression in previously suppressed subjects, providing proof of principle for a two-drug maintenance regimen using Cabotegravir and a non-nucleoside reverse transcriptase inhibitors (NNRTI).

A Long-acting injectable formulation of Cabotegravir has demonstrated prolonged exposures (≥30 days) following a single injection and has recently been approved as once monthly regimen for HIV treatment.

On the other hand, unplanned pregnancies account for nearly half of all pregnancies worldwide and lead to almost 100,000 maternal deaths per year as a result of unsafe abortions and complications of pregnancy and delivery (World Health Organization, 2007). While several long-acting parenteral contraceptives are available commercially, no long-acting therapies that prevent both pregnancy and HIV are currently available. Multipurpose Prevention Technologies (MPTs) that deliver combinations for prevention of HIV, and/or other sexually transmitted infections (STIs), as well as contraception are highly desirable.

Therefore, there is a need to prevent or treat HIV as well as prevent unplanned pregnancy particularly in many low-income regions and countries of the world, where HIV prevalence in women is high. Contraception is also important to prevent transmission of HIV to future generations and reduce infancy mortality rates.

### SUMMARY OF THE INVENTION

According to a first aspect of the invention there is provided a pharmaceutical composition comprising a compound of formula (I)

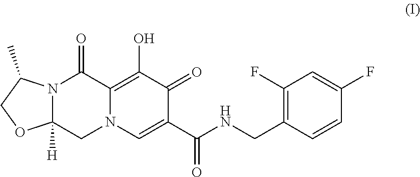 [SMILES: C[C@H]1CO[C@@H]2Cn3cc(C(=O)NCc4ccc(F)cc4F)c(=O)c(O)c3C(=O)N12]

or a pharmaceutically acceptable salt thereof and a contraceptive agent.

According to a second aspect of the invention, there is provided a combination comprising a compound of formula (I) or a pharmaceutically acceptable salt thereof and a contraceptive agent.

According to a third aspect of the invention, there is provided a method of preventing pregnancy and treating or preventing HIV in a human in need thereof comprising administering to said human a therapeutically effective amount of a pharmaceutical composition as defined herein.

According to a further aspect of the invention, there is provided a method of preventing pregnancy and treating or preventing HIV in a human in need thereof comprising administering to said human a therapeutically effective amount of a combination as defined herein.

The present invention also provides a pharmaceutical composition defined herein for use in the treatment or prevention of HIV.

The present invention also provides a combination as defined herein for use in the treatment or prevention of HIV.

In a final aspect, the present invention provides a kit comprising a compound of formula (I) and a contraceptive agent.

The present invention is advantageous in a number of respects. Specifically, the combination of a compound of formula (I) and a contraceptive agent may be safe, stable over extended period of time and effective to treat and/or prevent HIV as well as prevent pregnancy. A pharmaceutical composition according to the invention comprising of a compound of formula (I) and a contraceptive agent, particularly LNG or LNG-b may provide protection against HIV infection and prevent pregnancy for 2 to 3 months.

### DESCRIPTION OF DRAWINGS/FIGURES

FIG. 1 Pharmacokinetic profile of intramuscularly administered LNG (0.2 and 1 micron) co-formulated with Cabotegravir.

FIG. 2 Pharmacokinetic profile of LNG co-formulated with Cabotegravir after intramuscular and subcutaneous administration.

FIG. 3 Pharmacokinetic profile of LNG and LNG-b co-formulated with Cabotegravir after subcutaneous administration.

FIG. 4 Pharmacokinetic profile of Cabotegravir and LNG co-formulated with Cabotegravir blood concentrations after intramuscular administration.

### DETAILED DESCRIPTION OF THE INVENTION

### Definitions

As used herein, the term ‘pharmaceutical composition’ means a composition that is suitable for pharmaceutical use.

As used herein, the term “co-administer” refers to simultaneous or sequential administration such that therapeutically effective amounts of the compounds are both present in the body of the patient. The term “co-administer” also refers to administration at the same time, as part of a single formulation. Co-administration includes administration of pharmaceutical composition of compounds of formula (I) and contraceptives, for example, administration of a compound of formula (I) and a contraceptive within seconds, minutes, or hours of the administration of one another. For example, in some embodiments, a unit dose of one of a compound of formula (I) or a contraceptive is administered first, followed within seconds or minutes by administration of the other, by either the same or different routes

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see http://www.wiley.com/WileyCDA/WileyTitle/productCd-3906390519.html). Suitable pharmaceutically acceptable salts can include acid or base addition salts Suitable pharmaceutically acceptable salts of the invention include base addition salts.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

“Therapeutically effective amount” or “effective amount” refers to that amount of the compound being administered that will prevent a condition or will relieve to some extent one or more of the symptoms of the disorder being treated. Pharmaceutical compositions suitable for use herein include compositions wherein the active ingredients are contained in an amount sufficient enough to achieve the intended purpose. Determination of a therapeutically effective amount is well within the capability of those skilled in the art, especially in light of the detailed disclosure provided herein.

As used herein, the term “treatment” or “treating” in the context of therapeutic methods, refers to alleviating the specified condition, eliminating or reducing the symptoms of the condition, slowing or eliminating the progression, invasion, or spread of the condition and reducing or delaying the reoccurrence of the condition in a previously afflicted subject. The present invention further provides use of the compounds of the invention for the preparation of a medicament for the treatment of several conditions in a mammal (e.g., human) in need thereof.

As used herein, the term “prevention” or “preventing” in the context of therapeutic methods, refers to precluding the specified condition or symptoms of the condition, or in the occurrence of prior infection, precluding the re-occurrence of the condition. The present invention further provides use of the compounds of the invention for the preparation of a medicament for the prevention of several conditions in a mammal (e.g., human) in need thereof.

As used herein, the term “parenteral” or “parenterally” in the context of therapeutic methods, refers to a route of administration of a pharmaceutical compound or composition other than by oral administration. Parenteral routes of administration suitable for use herein include injection, infusion, implantation or some other route other than the alimentary canal. Parenteral routes of injection administration include intravenous, intramuscular and subcutaneous.

### STATEMENT OF THE INVENTION

In the present invention, the pharmaceutical composition or the combination may be used to treat or, alternatively, prevent HIV which unless further clarified is intended to mean HIV-1. As an alternative embodiment, the pharmaceutical compositions and combinations of the invention may also be effective against HIV-2, or against patients having dual HIV-1/HIV-2 infection. In addition, the pharmaceutical compositions and combinations of the present invention prevent pregnancy and the spread of sexually transmitted HIV.

According to a first aspect of the invention, the present invention provides a pharmaceutical composition comprising a compound of formula (I)

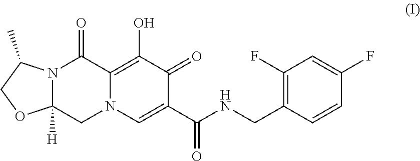 [SMILES: C[C@H]1CO[C@@H]2Cn3cc(C(=O)NCc4ccc(F)cc4F)c(=O)c(O)c3C(=O)N12]

or a pharmaceutically acceptable salt thereof and a contraceptive agent.

The compound of formula (I) is Cabotegravir. Cabotegravir (N-((2,4-Difluorophenyl)methyl)-6-hydroxy-3-methyl-5,7-dioxo-2,3,5,7,11,11a hexahydro(1,3)oxazolo(3,2-a)pyrido(1,2-d)pyrazine-8-carboxamide) is described in U.S. Pat. No. 8,129,385 and WO 2021/116872, incorporated herein by reference. Cabotegravir is an integrase strand transfer inhibitor (INSTI) that exhibits subnanomolar potency and antiviral activity against a broad range of HIV-1 strains. Oral administration of Cabotegravir has exhibited acceptable safety and tolerability profiles, a long half-life, and few drug-drug interactions. Cabotegravir has been demonstrated to be efficacious in treatment and prevention of HIV both in oral and parenteral dosage forms, see for instance, Margolis D A, Brinson C C, Eron J J, et al. 744 and Rilpivirine as Two Drug Oral Maintenance Therapy: LAI116482 (LATTE) Week 48 Results. 21st Conference on Retroviruses and Opportunistic Infections (CROI); Mar. 3-6, 2014; Boston, MA, Margolis D A, Podzamczer D, Stellbrink H-J, et al. Cabotegravir+Rilpivirine as Long-Acting Maintenance Therapy: LATTE-2 Week 48 Results. 21st International AIDS Conference; Jul. 18-22, 2016; Durban, South Africa, Abstract THAB0206LB. Levin: Conference reports for National AIDS Treatment Advocacy Project (NATAP); 2016, and Markowitz M, Frank I, Grant R, et al. ECLAIR: Phase 2A Safety and PK Study of Cabotegravir LA in HIV-Uninfected Men. Abstract presented at: 23rd Conference on Retroviruses and Opportunistic Infections (CROI); Feb. 22-25, 2016; Boston, MA.

In an embodiment of the invention, Cabotegravir is present in the pharmaceutical composition as the free acid. In an alternative embodiment of the invention, Cabotegravir is present as the sodium salt. In one embodiment of the invention, Cabotegravir is present as a prodrug.

The pharmaceutical compositions of the invention also comprise a contraceptive agent. The terms “contraceptive agent” and “contraceptive” are used interchangeably herein. For the purposes of this invention, the inventors focus on hormone level control compounds to prevent mature eggs from being released by the ovaries during ovulation or prevent fertilized eggs from implanting in the womb. Hormone regulating contraceptives come in a variety of forms including injectables, implants, vaginal rings, intrauterine devices, oral tablets and transdermal patches.

Preferably, the contraceptive agent used in the compositions of the present invention is a female contraceptive agent.

In one embodiment, the contraceptive is selected from the group consisting of progesterone, Norethisterone, Medroxyprogesterone 27-acetate (MPA), Levonorgestrel, Levonorgestrel-butanoate, Levonorgestrel cyclobutylcarboxylate, medroxyprogesterone acetate, Norgestrel (Levonorgestrel diastereomeric mix), Desogestrel, Gestodene, Norgestimate, Etonogestrel, Drospirenone and Dienogest.

In one embodiment, the contraceptive is Levonorgestrel (available from Asta Tech Inc., Bristol, PA). In one embodiment, the contraceptive is Levonorgestrel-butanoate (available from Pharmaron Inc., Louisville, KY). Levonorgestrel is a hormonal medication used in a number of birth control methods and has an established track record of safety and efficacy and is well suited for incorporation into controlled-release devices due to its low molecular weight, hydrophobicity, physical stability, and potency for example, it has been approved for delivery via subcutaneous implants and intrauterine systems. In one embodiment, the contraceptive is Levonorgestrel-butanoate, the prodrug of Levonorgestrel.

Levonorgestrel is represented by formula (II):

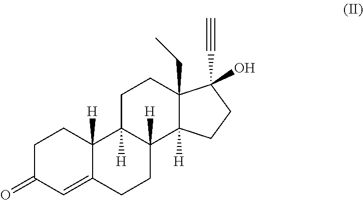

Levonorgestrel-butanoate is represented by formula (III):

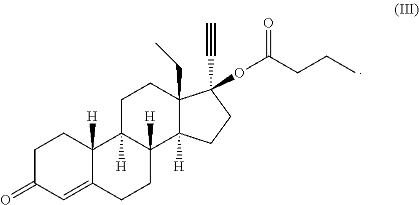

The pharmaceutical composition of the invention may further comprise a polyethylene glycol (PEG) and a poloxamer.

Poloxamers are nonionic triblock copolymers composed of a central hydrophobic chain of polyoxypropylene (polypropylene oxide) flanked by two hydrophilic chains of polyoxyethylene (polyethylene glycol). Poloxamers are represented by formula (IV). Poloxamers are also known by the trade names Synperonics, Pluronics, and Kolliphor. Poloxamers are commonly named with the letter “P” followed by three digits, the first two digits x 100 give the approximate molecular mass of the polyoxypropylene core, and the last digit x 10 gives the percentage polyoxyethylene content (e.g., P407=Poloxamer with a polyoxypropylene molecular mass of 4,000 g/mol and a 70% polyoxyethylene content).

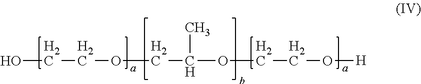

In an embodiment of the present invention, the poloxamer may be P237, P338 or P407. P237, P338 and P407 are commercially available. In an embodiment of the invention, the poloxamer is P237. In an embodiment the poloxamer is P407. In an embodiment the poloxamer is P338.

PEG is a polymer of ethylene oxide and is represented by formula (III):

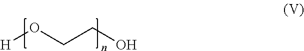

In embodiments of the invention, n may be any suitable number. In an embodiment of the invention PEG has a number average mean molecular weight (Mn) of from 1000 to 8000 g/mol. In an embodiment of the invention PEG has an Mₙ of from 2500 to 5000 g/mol. In an embodiment of the invention PEG has an Mₙ of from 3000 to 4000 g/mol. In an embodiment of the invention PEG has an Mₙ of 3100 to 3700 g/mol. In an embodiment of the invention, PEG is PEG 3350 i.e. PEG has an Mₙ of 3350 g/mol. PEG3350 is commercially available.

The combination of PEG and poloxamer may enable a high Cabotegravir and contraceptive concentration to be achieved. This enables the composition of the invention to treat HIV in a patient for up to 3 months, therefore enabling dosing once every month, 2 months or 3 months. Further, this enables the composition of the invention to prevent pregnancy for up to 3 months.

In an embodiment of the invention, the pharmaceutical composition comprises Cabotegravir, a contraceptive agent, polyethylene glycol 3350 and poloxamer 338. In another embodiment of the invention the pharmaceutical composition comprises Cabotegravir, Levonorgestrel, polyethylene glycol 3350 and poloxamer 338. In a further embodiment the pharmaceutical composition comprises Cabotegravir, Levonorgestrel-butanoate, polyethylene glycol 3350 and poloxamer 338.

In an embodiment, the pharmaceutical composition further comprises mannitol. In the pharmaceutical compositions of the present invention mannitol is used as a tonicity adjuster.

In an embodiment of the invention, the pharmaceutical composition comprises from 350 to 600 mg/mL of cabotegravir. In an embodiment of the invention, the pharmaceutical composition comprises from 350 to 500 mg/mL of cabotegravir. In another embodiment, the pharmaceutical composition comprises 380 to 420 mg/mL of cabotegravir. In another embodiment, the pharmaceutical composition comprises about 400 mg/mL of cabotegravir. In an alternative embodiment the pharmaceutical composition comprises about 500 mg/mL of cabotegravir.

In an embodiment, the pharmaceutical composition comprises about 200 mg/mL of Cabotegravir, as referenced by US 20170027933. In an embodiment, the pharmaceutical composition further comprises a polysorbate and polyethylene glycol. In an embodiment. the polysorbate is polysorbate and polyethylene glycol is polyethylene glycol 3350. In an embodiment the pharmaceutical composition further comprises mannitol.

In an embodiment the pharmaceutical composition comprises a contraceptive agent concentration of about 15 to 60 mg/mL. The pharmaceutical composition may comprise a contraceptive agent concentration of about 20 to 55 mg/mL. In one embodiment, the pharmaceutical composition comprises contraceptive agent concentration of 40 mg/mL. In another embodiment the pharmaceutical composition comprises a contraceptive agent of about 50 mg/mL. In some embodiments the contraceptive agent is Levonorgestrel and it is present as the concentrations discussed above. In other embodiments the contraceptive agent is Levonorgestrel-butanoate and it is present at the concentrations discussed above.

In embodiments of the invention, Cabotegravir and the contraceptive agent are present in the pharmaceutical composition in the form of particles. Desired particle sizes may be achieved by any suitable means. In an embodiment of the invention, desired particle sizes are achieved by wet bead milling. Median particle diameter may be measured by any suitable means, for example laser diffraction.

In one embodiment, the median particle diameter of the contraceptive agent is about 0.2 to 1.0 μm. In another embodiment the median particle diameter of the contraceptive agent is 0.2 μm. Pharmaceutical compositions of the present invention may retain their particle size over time. This is advantageous as increase in particle size can lead to poor resuspension. Poor resuspension can, for example, make it difficult for healthcare professionals to withdraw a pharmaceutical composition from, for example, a vial or flask. Particle size stability is believed to be important for resuspension of the composition over time and reduces the risk of changing pharmacokinetics.

According to a second aspect of the invention, the present invention provides a combination comprising a compound of formula (I) or a pharmaceutically acceptable salt thereof and a contraceptive agent.

The compound of formula (I) is as described above in the first aspect.

In one embodiment, the combination comprises a compound of formula (I) is in the form of a sodium salt. In another embodiment, the compound of formula (I) is in the form of a free acid.

The combination comprises a contraceptive agent. The contraceptive agent is as described in the first aspect. In one embodiment, the contraceptive is selected from the group consisting of progesterone, Norethisterone, Medroxyprogesterone 27-acetate (MPA), Levonorgestrel, Levonorgestrel-butanoate, Levonorgestrel cyclobutylcarboxylate, medroxyprogesterone acetate, Norgestrel (Levonorgestrel diastereomeric mix), Desogestrel, Gestodene, Norgestimate, Etonogestrel, Drospirenone and Dienogest.

In one embodiment, the contraceptive is Levonorgestrel. In another embodiment, the contraceptive is Levonorgestrel-butanoate.

According to a third aspect, the present invention provides a method of preventing pregnancy and treating or preventing HIV in a human in need thereof comprising administering to said human a therapeutically effective amount of a pharmaceutical composition as defined herein.

The pharmaceutical composition may be administered by any suitable means. In one embodiment, the method comprises administering the pharmaceutical composition parenterally. The pharmaceutical composition may be administered in the form of an injectable. In an embodiment the pharmaceutical composition is administered intramuscularly. In an one embodiment the pharmaceutical composition is administered subcutaneously. In one embodiment, the pharmaceutical composition is administered as a single injection. In another embodiment, the pharmaceutical composition is administered in multiple injections. In one embodiment, the pharmaceutical composition is administer as a fixed dose.

In another embodiment, the method comprises administering the pharmaceutical composition to a human once every month. In another embodiment, the method comprises administering the pharmaceutical composition to a human once every 2 months. In another embodiment, the method comprises administering the pharmaceutical composition to a human once every 3 months.

According to a further aspect of the invention, the present invention provides a method of preventing pregnancy and treating or preventing HIV in a human in need thereof comprising administering to said human a therapeutically effective amount of a combination, wherein the combination comprises a compound of formula (I) and a contraceptive as defined herein.

In one embodiment, the method comprises administering about 1 mL to about 3 mL of the combination to the human. In another embodiment, the method comprises administering 1 mL of the combination to the human. In another embodiment, the method comprises administering 2 mL of the combination to the human. In another embodiment, the method comprises administering 3 mL of the combination to the human.

The combination may be administered via any suitable means. In one embodiment, the method comprises administering the combination parenterally. In an embodiment the combination is administered intramuscularly. In an embodiment the combination is administered subcutaneously.

In one embodiment, the method comprises administering the combination to a human once every month. In another embodiment, the method comprises administering the combination to a human once every 2 months. In another embodiment, the method comprises administering the combination to a human once every 3 months.

In one embodiment, the combination may be administered together or separately. In one embodiment, the method comprises administering the combination to a human separately. In another embodiment, the method comprises administering the combination to a human simultaneously. In another embodiment, the method comprises administering the combination to a human sequentially.

In an embodiment, the method comprises administering the combination is self-administered by the human. The term “self-administered”, as used herein, means administration by someone other than a healthcare professional, for example, a patient may administer the pharmaceutical composition to themselves, or someone else, other than a healthcare professional may administer the pharmaceutical composition to the patient. In another embodiment, the method comprises administering the combination is administered by a health-care professional.

According to a further aspect of the invention, the present invention provides a pharmaceutical composition as defined herein, for use treatment or prevention of HIV.

In one embodiment, the pharmaceutical composition for use, is suitable for the use in the prevention of pregnancy. As discussed above, the pharmaceutical compositions of the present invention comprise a contraceptive agent therefore can be used to prevent pregnancy.

The pharmaceutical composition may be administered by any suitable means. In one embodiment, the pharmaceutical composition is administered parenterally. The pharmaceutical composition may be administered in the form of an injectable. In an embodiment the pharmaceutical composition is administered intramuscularly. In an embodiment the pharmaceutical composition is administered subcutaneously.

In one embodiment, the pharmaceutical composition for use is administered once every month. In another embodiment, the pharmaceutical composition for use is administered once every 2 months. In another embodiment, the pharmaceutical composition for use is administered once every 3 months.

According to a further aspect of the invention, the present invention provides a combination as defined herein for use in the treatment or prevention of HIV.

In an embodiment, the combination for use is suitable for the prevention of pregnancy.

As discussed above, the combination of the present invention comprises a contraceptive agent therefore can be used to prevent pregnancy.

The combination may be administered by any suitable means. In one embodiment, the combination is administered parenterally. The combination may be administered in the form of an injectable. In an embodiment the combination is administered intramuscularly. In an embodiment the combination is administered subcutaneously.

In one embodiment, the combination is administered once every month. In another embodiment, the combination is administered once every 2 months. In another embodiment, the combination is administered once every 3 months.

According to a final aspect of the invention, the present invention provides a kit, wherein the kit comprises a compound of formula (I) or a pharmaceutically acceptable salt thereof, and a contraceptive agent. In one embodiment, the kit comprises a syringe comprising the pharmaceutical composition of the invention as well as a leaflet comprising use instructions.

The following non-limiting Examples illustrate the present invention.

### EXAMPLES

### Example 1: Pharmaceutical Composition

Cabotegravir at 500 mg/mL:

A formulation vehicle was prepared by dissolving 11.56 g Poloxamer 338 (BASF), 4.63 g mannitol (Roquette Freres), and 11.56 g PEG3350 (Clariant) in 203.54 g water for injection (WFI) and filtering the solution through a 0.2 μm filter. The formulation vehicle was added to Cabotegravir (free acid) to prepare a 500 mg/ml coarse suspension. The coarse suspension, while stirred, was circulated through a wet bead mill set at 29.7 Hz (Netzsch MiniCer) containing 5 0.30 mm YTZ grinding beads (Nikkato Corp) at 73-145 ml/min until the desired median particle diameter of 0.2 to 1.0 μm was reached as measured by laser diffraction using a Mastersizer 3000 by Malvern. The wet bead mill was cooled to maintain a temperature between 1 and 25° C.

Formulation of Cabotegravir at 200 mg/mL

Cabotegravir suspension at 500 mg/mL was diluted to 200 mg/mL using a solution of 2% Mannitol in water to adjust the final concentrations to target. Concentrations of P338 and PEG3350 were at 2% in the final formulation. The resulting suspensions were flushed with nitrogen, stoppered (FM457 stopper), and sealed. The suspensions were terminally sterilized by gamma irradiation at a minimum dose of 25 kGy

### Levonorgestrel (LNG) and Levonorgestrel-Butanoate (LNGb)

LNG and LNGb formulations were prepared using the same vehicle as described above for Cabotegravir. The formulation vehicle was added to LNG or LNGb to prepare formulations between 200 and 350 mg/mL coarse suspension. The coarse suspension, while stirred, was circulated through a wet bead mill set at 29.7 Hz (Netzsch MiniCer) containing 5 0.30 mm YTZ grinding beads (Nikkato Corp) at 73-145 ml/min until the desired median particle diameter of 0.2 to 1.0 μm was reached as measured by laser diffraction using a Mastersizer 3000 by Malvern. The wet bead mill was cooled to maintain a temperature between 1 and 25° C.

### Co-Formulation of Cabotegravir and LNG or LNGb

To prepare the combination product, LNG and LNG-b suspensions were added to Cabotegravir suspensions at 500 mg/mL to achieve a final Cabotegravir concentration at approximately 400 mg/mL and a final LNG and LNGb concentration of 15-50 mg/mL, using a solution of 2.0% Mannitol in water to adjust the final concentrations to target. Concentrations of P338 and PEG3350 ranged from 4.5-8.5%. The resulting suspensions were flushed with nitrogen, stoppered (FM457 stopper), and sealed. The suspensions were terminally sterilized by gamma irradiation at a minimum dose of 25 kGy.

### Example 2: Stability

Formulations 1 to 4 (shown in Table 1) were made, as defined above, and were stored under accelerated stability conditions (30° C./65% RH) for 6 months. Particle size measurements was performed using laser diffraction (Malvern Mastersizer 3000). This method generates particle size distributions for the samples which can be used monitor any potential physical stability issues that can arise from particle size growth over time. Table 2 shows minimal particle size growth of formulations over 6 months. Table 3 shows particle size reporting definitions.

TABLE 1
Formulations

| | |
|---|---|
| Formu- | 400 mg/mL CAB/50 mg/mL LNG at 0.2 um in 4.5% P338, |
| lation 1 | 4.5% PEG3350 and 2.0% Mannitol - 6 months stability |
| Formu- | 400 mg/mL CAB/50 mg/mL LNG at 0.2 um in 8.5% P338, |
| lation 2 | 8.5% PEG3350 and 2.0% Mannitol - 6 months stability |
| Formu- | 400 mg/mL CAB/50 mg/mL LNGb at 0.2 um in 4.5% P338, |
| lation 3 | 4.5% PEG3350 and 2.0% Mannitol - 6 months stability |
| Formu- | 400 mg/mL CAB/50 mg/mL LNGb at 0.2 um in 8.5% P338, |
| lation 4 | 4.5% PEG3350 and 2.0% Mannitol - 6 months stability |

TABLE 2
Particle size stability for CAB/LNG and CAB/LNGb coformulations
up to 6 months under stress conditions (30° C./65% RH)

| Particle | | Formu- | Formu- | Formu- | Formu- |
|---|---|---|---|---|---|
| Size | Time Point | lation 1 | lation 2 | lation 3 | lation 4 |
| X10 | t = 0 | 0.14 | 0.12 | 0.14 | 0.13 |
| | t = 1 month | 0.13 | 0.13 | 0.13 | 0.13 |
| | t = 3 months | 0.12 | 0.13 | 0.12 | 0.12 |
| | t = 6 months | 0.10 | 0.10 | 0.10 | 0.09 |
| X50 | t = 0 | 0.30 | 0.27 | 0.31 | 0.28 |
| | t = 1 month | 0.29 | 0.28 | 0.27 | 0.27 |
| | t = 3 months | 0.25 | 0.28 | 0.26 | 0.27 |
| | t = 6 months | 0.25 | 0.26 | 0.24 | 0.24 |
| X90 | t = 0 | 0.64 | 0.60 | 0.67 | 0.61 |
| | t = 1 month | 0.62 | 0.61 | 0.57 | 0.59 |
| | t = 3 months | 0.56 | 0.61 | 0.59 | 0.64 |
| | t = 6 months | 0.63 | 0.63 | 0.50 | 0.60 |

TABLE 3
Particle size reporting definitions

| | |
|---|---|
| X50 | Median particle diameter (microns); here used on a volumetric |
| | basis, i.e. 50% by volume of the particles is smaller than this |
| | diameter and 50% is larger |
| X10 | Particle diameter corresponding to 10% cumulative undersize |
| | distribution; here by volume (microns) |
| X90 | Particle diameter corresponding to 90% of the cumulative |
| | undersize distribution; here by volume (microns) |

### Example 3: Pharmacokinetic Evaluation

### Co-Formulation of Cabotegravir and LNG or LNGb for Suspensions Used in PK Studies

To prepare the combination product, LNG and LNG-b suspensions were added to Cabotegravir suspensions at 500 mg/mL to achieve a final Cabotegravir concentration of approximately 200 mg/mL and a final LNG and LNGb concentration of 50 mg/mL, using a solution of 2.0% Mannitol in water to adjust the final concentrations to target. Concentrations of P338 and PEG3350 were at 4.5%. The resulting suspensions were flushed with nitrogen, stoppered (FM457 stopper), and sealed. The suspensions were terminally sterilized by gamma irradiation at a minimum dose of 25 kGy.

The pharmacokinetics of co-suspensions of Cabotegravir and LNG/LNG-b, prepared as described above, were evaluated in male Sprague Dawley rats. The formulations that were tested included:
i. 200 mg/mL Cabotegravir and 40 mg/mL LNG—0.2 microns
ii. 200 mg/mL Cabotegravir 40 mg/mL LNG—1 micron
iii. 200 mg/mL Cabotegravir and 40 mg/mL LNGb—0.2 microns
iv. 200 mg/mL Cabotegravir 40 mg/mL LNGb—1 micron

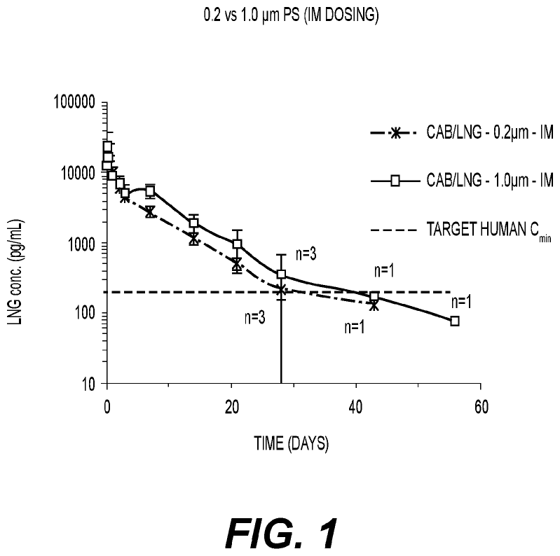

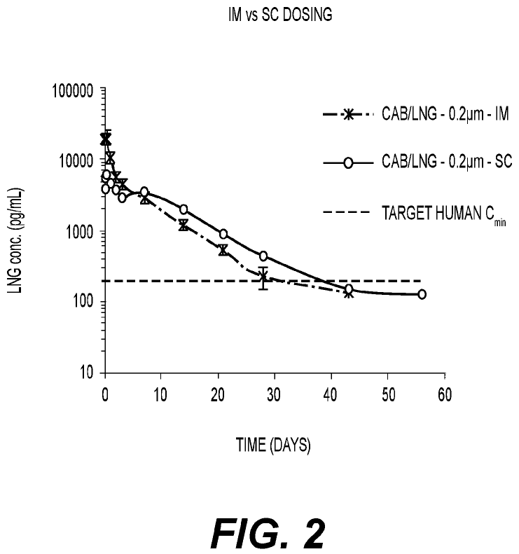

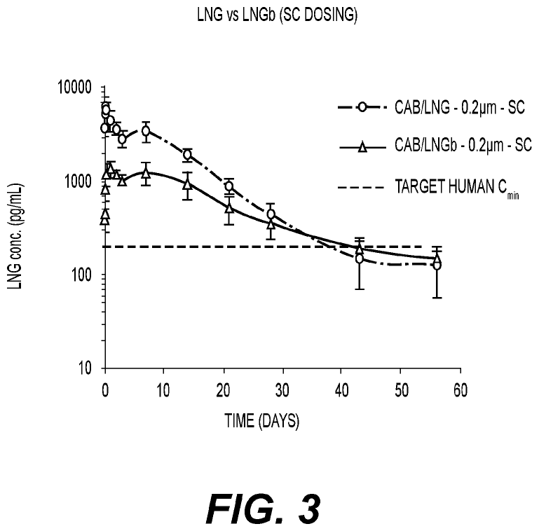

Rats were administered co-suspensions either intramuscularly or subcutaneously at target doses of 10 mg/kg Cabotegravir and 50 mg/kg LNG or LNGb. FIG. 1 shows pharmacokinetic profiles relative to Levonorgestrel blood concentration from Cabotegravir-LNG co-formulations with two different particle sizes of LNG (0.2 and 1 micron (×50)) after intramuscular administration. Based on this data, there was no real impact of particle size over the two-month period evaluated. FIG. 2 is a comparison of Levonorgestrel blood concentration pharmacokinetic profiles after intramuscular vs. subcutaneous administration. The average particle size of LNG for these formulations was 0.2 microns in diameter. Based on this data, there does not appear to be a significant impact on PK of intramuscular vs. subcutaneous dosing. Furthermore, FIG. 3 is a comparison of LNG and LNG-b co-formulated with Cab. As shown, LNGb yielded a profile with a lower Cmax and a slightly longer apparent half-life.

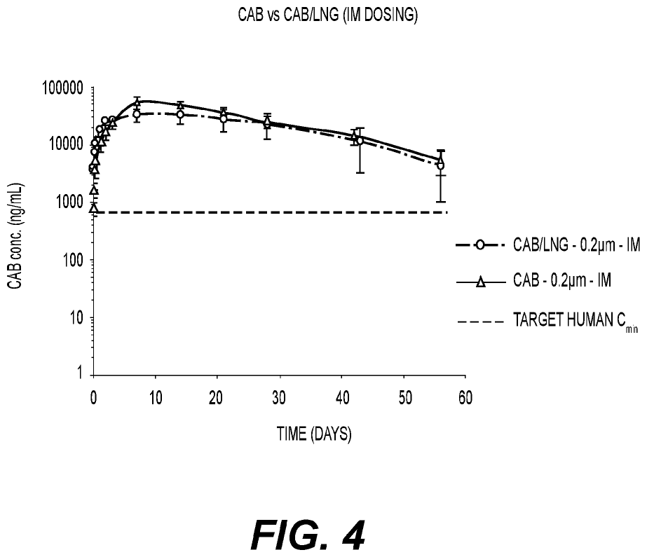

FIG. 4 shows the pharmacokinetic profiles as blood concentrations of Cabotegravir post-intramuscular administration of the single agent—Cabotegravir Long-Acting suspension, as compered to Cabotegravir-LNG co-formulation (both at average particles size of 0.2 micron (×50)). No clear difference is evident throughout the study duration, indicating that the simultaneous administration of the two agents does not impact the pharmakokinetic profile of Cabotegravir in the Rat.

It will be understood that the present invention has been described above purely by way of example, and modification of detail can be made within the scope of the invention. Each feature disclosed in the description, and where appropriate the claims and drawings may be provided independently or in any appropriate combination.

1. A pharmaceutical composition comprising a compound of formula (I) 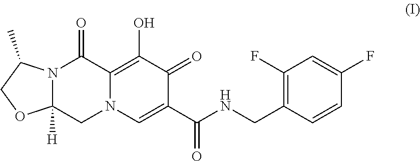 [SMILES: C[C@H]1CO[C@@H]2Cn3cc(C(=O)NCc4ccc(F)cc4F)c(=O)c(O)c3C(=O)N12]
or a pharmaceutically acceptable salt thereof
and a contraceptive agent.

2. The pharmaceutical composition according to claim 1, wherein the contraceptive agent is selected from the group consisting of progesterone, Norethisterone, Medroxyprogesterone 27-acetate (MPA), Levonorgestrel, Levonorgestrel-butanoate, Levonorgestrel cyclobutylcarboxylate, Medroxyprogesterone acetate, Norgestrel (Levonorgestrel diastereomeric mix), Desogestrel, Gestodene, Norgestimate, Etonogestrel, Drospirenone and Dienogest.

3. The pharmaceutical composition according to claim 2, wherein the contraceptive agent is Levonorgestrel.

4. The pharmaceutical composition according to claim 2, wherein the contraceptive agent is Levonorgestrel-butanoate.

5. The pharmaceutical composition according to claim 1, wherein the pharmaceutical composition further comprises a polyethylene glycol and a poloxamer.

6. The pharmaceutical composition according to claim 5, wherein the polyethylene glycol is polyethylene glycol 3350 and the poloxamer is poloxamer 338.

7. The pharmaceutical composition according to claim 5, wherein the pharmaceutical composition further comprises mannitol.

8. The pharmaceutical composition according to claim 1, wherein the compound of formula (I) or a pharmaceutically acceptable salt thereof is present in a concentration of about 350 mg/mL to 600 mg/mL.

9. The pharmaceutical composition according to claim 8, wherein the compound of formula (I) or a pharmaceutically acceptable salt thereof, is present in a concentration of about 400 mg/mL.

10. The pharmaceutical composition according to claim 1, wherein the pharmaceutical composition further comprises a polysorbate and polyethylene glycol.

11. The pharmaceutical composition according to claim 10, wherein the polysorbate is polysorbate 20 and polyethylene glycol is polyethylene glycol 3350.

12. The pharmaceutical composition according to claim 10, wherein the pharmaceutical composition further comprises mannitol.

13. The pharmaceutical composition according to claim 10, wherein the compound of formula (I) or a pharmaceutically acceptable salt thereof is present in a concentration of about 200 mg/mL.

14. The pharmaceutical composition according to claim 1, wherein the contraceptive agent is present in a concentration of about 15 to 60 mg/mL.

15. The pharmaceutical composition according to claim 14, wherein the contraceptive agent is present in a concentration of about 50 mg/mL.

16. The pharmaceutical composition according to claim 1, wherein the contraceptive agent is present in the form of particles with a median particle diameter of about 0.2 to 1.0 μm.

17. The pharmaceutical composition according to claim 16, wherein the contraceptive agent is present in the form of particles with a median particle diameter of 0.2 μm.

18. A combination comprising a compound of formula (I), 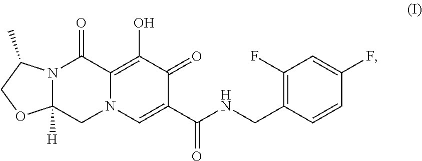 [SMILES: C[C@H]1CO[C@@H]2Cn3cc(C(=O)NCc4ccc(F)cc4F)c(=O)c(O)c3C(=O)N12]
or a pharmaceutically acceptable salt thereof,
and a contraceptive agent.

19. The combination according to claim 18, wherein the compound of formula (I) is in the form of a sodium salt.

20. The combination according to claim 18, wherein the compound of formula (I) is in the form of a free acid.

21. The combination according to claim 18, wherein the contraceptive agent is selected from the group consisting of progesterone, Norethisterone, Medroxyprogesterone 27-acetate (MPA), Levonorgestrel, Levonorgestrel-butanoate, Levonorgestrel cyclobutylcarboxylate, Medroxyprogesterone acetate, Norgestrel (Levonorgestrel diastereomeric mix), Desogestrel, Gestodene, Norgestimate, Etonogestrel, Drospirenone and Dienogest.

22. The combination according to claim 21, wherein the contraceptive agent is Levonorgestrel.

23. The combination according to claim 21, wherein the contraceptive agent is Levonorgestrel-butanoate.

24. A method of preventing pregnancy and treating or preventing HIV in a human in need thereof comprising administering to said human a therapeutically effective amount of the pharmaceutical composition according to claim 1.

25. The method according to claim 24, wherein the method comprises administering the pharmaceutical composition parenterally.

26. The method according to claim 24, wherein the method comprises administering the pharmaceutical composition to the human once every month, once every 2 months or once every 3 months.

27. A method of preventing pregnancy and treating or preventing HIV in a human in need thereof comprising administering to said human a therapeutically effective amount of the combination according to claim 18.

28. The method according to claim 27, wherein the combination is administered parenterally.

29. The method according to claim 27, wherein the method comprises administering the combination to the human once every month, once every 2 months or once every 3 months.

30. The method according to claim 27, wherein the method comprises administering the combination to the human simultaneously or sequentially.

31. (canceled)

32. (canceled)

33. (canceled)

34. (canceled)

35. (canceled)

36. (canceled)

37. A kit comprising a compound of formula (I), 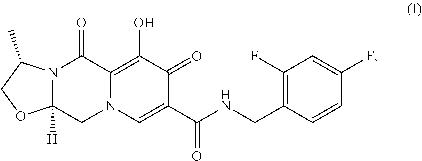 [SMILES: C[C@H]1CO[C@@H]2Cn3cc(C(=O)NCc4ccc(F)cc4F)c(=O)c(O)c3C(=O)N12]
or a pharmaceutically acceptable salt thereof,
and a contraceptive agent.

38. The pharmaceutical composition according to claim 2, wherein the contraceptive agent is medroxyprogesterone acetate.

39. The combination according to claim 21, wherein the contraceptive agent is medroxyprogesterone acetate.
